(19) United States
(12) Patent Application Publication
(10) Pub. No.: US 2015/0191506 A1
(43) Pub. Date: Jul. 9, 2015

(54) ADSORBENT CONSISTING OF CARRIER WHICH BOUND WITH POLYPEPTIDE COMPRISING B-DOMAIN MUTANT DERIVED FROM PROTEIN A

(71) Applicants: ASAHI KASEI MEDICAL CO., LTD., Tokyo (JP); NOMADIC BIOSCIENCE CO., LTD., Okayama (JP)

(72) Inventors: Kazuo Okuyama, Tokyo (JP); Ichiro Koguma, Tokyo (JP); Tomokiyo Marumoto, Tokyo (JP); Satoshi Sato, Okayama (JP)

(73) Assignees: ASAHI KASEI MEDICAL CO., LTD., Tokyo (JP); NOMADIC BIOSCIENCE CO., LTD., Okayama (JP)

(21) Appl. No.: 14/410,616

(22) PCT Filed: Jun. 28, 2013
(86) PCT No.: PCT/JP2013/067865

§ 371 Date: Dec. 23, 2014

(30) Foreign Application Priority Data
Jun. 29, 2012 (JP) 2012-146610

(51) Int. Cl. C07K 1/22; B01J 20/24; C08F 16/06; C07K 16/00; C07K 14/195

(52) U.S. Cl. CPC C07K 1/22; C07K 16/00; C07K 14/195; C08F 16/06; B01J 20/24

(57) ABSTRACT

It is an object of the present invention to provide an affinity chromatographic adsorbent using temperature-responsive protein A, wherein the adsorbent is capable of improving the culture productivity of the temperature-responsive protein A and the stability of the temperature-responsive protein A in cell disruption solution. According to the present invention, there is provided an adsorbent consisting of a carrier, to which a polypeptide comprising a tag peptide, a linker sequence, and a B-domain mutant derived from protein A from the N-terminal side thereof binds, wherein the linker sequence is an amino acid sequence that does not comprise a Val-Pro-Arg sequence and is composed of 7 to 12 amino acid residues; and the binding property of the B-domain mutant derived from protein A to an immunoglobulin can vary depending on temperature under conditions of pH 5 to 9 and a temperature of lower than 60° C.

### TECHNICAL FIELD

The present invention relates to an adsorbent consisting of a carrier, to which a polypeptide comprising a B-domain mutant derived from protein A binds, wherein the binding property of the B-domain mutant derived from protein A to an immunoglobulin can vary depending on temperature. The adsorbent of the present invention can be used for purification of an immunoglobulin.

### BACKGROUND ART

The term “immunoglobulin” is a generic term used to refer to an antibody which recognizes a foreign matter entering a living body and then causes an immune reaction, and a polypeptide structurally or functionally similar to the antibody. The immunoglobulin includes IgG, IgM, IgA, IgD, and IgE. The immunoglobulin is useful in the field of life science studies, medicaments, clinical inspections, etc. As a method for producing a high-purity immunoglobulin, affinity chromatography has been applied. As ligands for affinity chromatography used for purification of an immunoglobulin, protein A derived from Staphylococcus having extremely high specificity and affinity to a common region in immunoglobulins (hereinafter referred to as “protein A”) and an immunoglobulin-binding domain thereof have been known. Protein A has been widely used in the process of producing antibody drugs. In the case of a conventionally known affinity chromatographic adsorbent comprising, as a ligand, protein A or a portion thereof (hereinafter referred to as a “conventional protein A adsorbent”), the adsorbed IgG needs to be eluted in an acidic range (pH 3 to 4). Thus, the conventional protein A adsorbent has been problematic in that a change in the three-dimensional structure of the purified IgG, association, aggregation, etc. would occur and the adsorbent would become inactivated.

As a means for solving this problem, a temperature-sensitive mutant derived from protein A that enables elution of the adsorbed IgG in a neutral range by controlling the affinity thereof for the IgG by temperature change has been proposed (hereinafter referred to as “temperature-responsive protein A”) (Patent Literature 1). However, an affinity chromatographic adsorbent using this temperature-responsive protein A (hereinafter referred to as a “temperature-responsive protein A adsorbent”) is not sufficient in terms of performance such as IgG-adsorbing capacity, when compared with the conventional protein A adsorbent. Hence, it has been strongly desired to improve the performance of the temperature-responsive protein A adsorbent.

The conventional protein A adsorbent has also been problematic in terms of expensiveness. Using such conventional protein A adsorbent, antibody drugs become extremely expensive, and this would cause increased pressure on the insurance finance. It is an important object to provide a temperature-responsive protein A adsorbent at a cost lower than the conventional protein A adsorbent. According to Patent Literature 1, the temperature-responsive protein A is produced as a polypeptide having a His-Tag sequence at the N-terminus thereof by culturing genetically recombinant Escherichia coli. However, the amount of such a polypeptide produced by culture is low, and the stability thereof in a cell disruption solution is also low. Hence, an expensive protease inhibitor must be used. Accordingly, it has been strongly desired to improve the culture productivity and stability of the temperature-responsive protein A.

### PRIOR ART LITERATURES

### Patent Literature

Patent Literature 1: International Publication W02008/143199

### SUMMARY OF INVENTION

### Object to be Solved by the Invention

It is an object to be solved by the present invention to provide an affinity chromatographic adsorbent using temperature-responsive protein A, wherein the adsorbent is capable of improving the culture productivity of the temperature-responsive protein A and the stability of the temperature-responsive protein A in cell disruption solution. It is another object of the present invention to provide an affinity chromatographic adsorbent using temperature-responsive protein A, wherein the adsorbent has an improved IgG-adsorbing capacity.

### Means for Solution of Object

As a result of intensive studies directed towards achieving the aforementioned objects, the present inventors have found that, in a polypeptide comprising a tag peptide, a linker sequence, and a B-domain mutant derived from protein A from the N-terminal side thereof, the culture productivity of the aforementioned polypeptide and the stability of the aforementioned polypeptide in cell disruption solution can be improved by optimizing the linker sequence which connects the tag peptide with the B-domain mutant derived from protein A, thereby completing the present invention.

Thus, the present invention provides the following.
(1) An adsorbent consisting of a carrier, to which a polypeptide comprising a tag peptide, a linker sequence, and a B-domain mutant derived from protein A from the N-terminal side thereof binds, wherein

the linker sequence is an amino acid sequence that does not comprise a Val-Pro-Arg sequence and is composed of 7 to 12 amino acid residues; and

the binding property of the B-domain mutant derived from protein A to an immunoglobulin can vary depending on temperature under conditions of pH 5 to 9 and a temperature of lower than 60° C.
(2) The adsorbent according to (1), wherein the linker sequence comprises 1 to 4 glycine residues and 3 to 7 serine residues.
(3) The adsorbent according to (1) or (2), wherein the linker sequence comprises a methionine residue.
(4) The adsorbent according to any one of (1) to (3), wherein the linker sequence comprises a leucine residue.
(5) The adsorbent according to any one of (1) to (4), wherein the linker sequence comprises a histidine residue.
(6) The adsorbent according to any one of (1) to (5), wherein the linker sequence is any one of:

an amino acid sequence composed of a glycine residue, a serine residue and a methionine residue;

an amino acid sequence composed of a glycine residue, a serine residue, a methionine residue and a histidine residue;

an amino acid sequence composed of a glycine residue, a serine residue, a methionine residue, a histidine residue and a leucine residue; and

an amino acid sequence composed of a glycine residue, a serine residue, a methionine residue, a histidine residue, a leucine residue and an arginine residue.
(7) The adsorbent according to any one of (1) to (6), wherein the linker sequence is an amino acid sequence shown by Ser-Ser-Gly-(Xaa)n-Met (wherein n represents an integer of 3 to 8, and an n number of Xaa each independently represents a glycine residue, a serine residue, a histidine residue, a leucine residue or an arginine residue).
(8) The adsorbent according to any one of (1) to (7), wherein the linker sequence is an amino acid sequence shown by Ser-Ser-Gly-Leu-(Xbb)m-His-Met (wherein m represents an integer of 1 to 6, and an m number of Xbb each independently represents a glycine residue, a serine residue or an arginine residue).
(9) The adsorbent according to any one of (1) to (8), wherein the tag peptide is a 6 x histidine tag.
(10) The adsorbent according to any one of (1) to (9), wherein the B-domain mutant derived from protein A comprises, in a single molecule thereof, at least one amino acid sequence having homology of 60% or more with the polypeptide of SEQ ID NO: 1 (with the proviso that at least Gly at position 19 and/or Gly at position 22 are substituted with Ala or Leu in the amino acid sequence shown in SEQ ID NO: 1), wherein the binding property of the amino acid sequence to an immunoglobulin can vary depending on temperature under conditions of pH 5 to 9 and a temperature of lower than 60° C.
(11) The adsorbent according to any one of (1) to (10), wherein the B-domain mutant derived from protein A comprises, in a single molecule thereof, at least one of the amino acid sequence shown in SEQ ID NO: 2.
(12) The adsorbent according to any one of (1) to (11), wherein the carrier is a particulate matrix for chromatography.
(13) The adsorbent according to any one of (1) to (12), wherein the mean particle diameter of the carrier is 20 to 200 μm.
(14) The adsorbent according to any one of (1) to (13), wherein the carrier is composed of a crosslinked polymer of polyvinyl alcohol.
(15) The adsorbent according to any one of (1) to (14), wherein the polypeptide is bound to the carrier via an amide bond.
(16) The adsorbent according to any one of (1) to (15), wherein the polypeptide binds to the carrier at a level of 20 mg/mL resin or more.
(17) The adsorbent according to any one of (1) to (16), wherein the maximum binding capacity of the immunoglobulin is 20 mg/mL resin or more.
(18) The adsorbent according to any one of (1) to (17), wherein the carrier comprises a carboxyl group at a level of 400 to 600 μmol/mL resin.
(19) The adsorbent according to any one of (1) to (11), wherein the carrier is a membrane.
(20) The adsorbent according to (19), wherein the membrane is a hollow fiber membrane.
(21) The adsorbent according to (19) or (20), wherein the membrane is produced from a base membrane into which a graft polymer chain is introduced.
(22) A method for purifying an immunoglobulin, which comprises allowing a sample containing the immunoglobulin to come into contact with the adsorbent according to any one of (1) to (21).

### Advantageous Effects of Invention

According to the present invention, a polypeptide comprising a B-domain mutant derived from protein A can be improved in terms of culture productivity, and it can also be improved in terms of stability in a cell disruption solution. Accordingly, temperature-responsive protein A can be provided at a low cost. Moreover, in the adsorbent of the present invention consisting of a carrier, to which a polypeptide comprising a B-domain mutant derived from protein A binds, the IgG-adsorbing capacity could be improved. Therefore, according to the present invention, an IgG purification process, which is more efficient and highly economical, can be provided.

### EMBODIMENTS FOR CARRYING OUT THE INVENTION

Hereinafter, the present invention will be described more in detail.

The adsorbent of the present invention consists of a carrier, to which a polypeptide comprising a tag peptide, a linker sequence, and a B-domain mutant derived from protein A from the N-terminal side thereof binds.

Examples of the tag peptide used in the present invention include known tags such as a tag composed of 2 to 6 histidine residues (His tag or 6× His), a tag composed of glutathione-S-transferase (GST tag), a maltose-bound polypeptide (MBP) tag, a calmodulin, Myc tag (c-myc tag), a FLAG-tag, or a green fluorescent protein GFP). Among these tags, a His tag, a GST tag, and the like are preferable. Since the size of the His tag is small, it has low immunogenicity, and thus, when the His tag is used, the purified polypeptide can be used without removing the tag. In addition, with regard to the His tag, a plasmid into which the His tag gene has previously been introduced is commercially available, and thus, it can be easily obtained.

The linker sequence used in the present invention does not contain a Val-Pro-Arg sequence, and it is an amino acid sequence composed of 7 to 12 amino acid residues.

One feature of the linker sequence used in the present invention is that the linker sequence does not contain the Val-Pro-Arg sequence that is a thrombin recognition sequence. By excluding the Val-Pro-Arg sequence from the linker sequence, stability during the production of the polypeptide can be improved, and thereby, culture productivity can also be improved. Moreover, stability when used can also be improved, and elution of a tag peptide such as a His tag can be prevented.

Another feature of the linker sequence used in the present invention is that the linker sequence is composed of 7 to 12 amino acid residues. The present invention has revealed that, when the number of amino acid residues constituting the linker sequence is 6 or less, or 13 or more, the expression level of the polypeptide is reduced, and thus that sufficient culture productivity cannot be achieved.

Preferably, the linker sequence may comprise 1 to 4 glycine residues and 3 to 7 serine residues. More preferably, the linker sequence may comprise one to three types of amino acid residues selected from a methionine residue, a leucine residue and a histidine residue.

Specific examples of the amino acid sequence of the above-described preferred linker sequence include:

an amino acid sequence composed of a glycine residue, a serine residue and a methionine residue;

an amino acid sequence composed of a glycine residue, a serine residue, a methionine residue and a histidine residue;

an amino acid sequence composed of a glycine residue, a serine residue, a methionine residue, a histidine residue and a leucine residue; and

an amino acid sequence composed of a glycine residue, a serine residue, a methionine residue, a histidine residue, a leucine residue and an arginine residue.

The linker sequence is even more preferably an amino acid sequence shown by Ser-Ser-Gly-(Xaa)n-Met (wherein n represents an integer of 3 to 8, and an n number of Xaa each independently represents a glycine residue, a serine residue, a histidine residue, a leucine residue or an arginine residue), and it is particularly preferably an amino acid sequence shown by Ser-Ser-Gly-Leu-(Xbb)m-His-Met (wherein m represents an integer of 1 to 6, and an m number of Xbb each independently represents a glycine residue, a serine residue or an arginine residue).

The binding property of the B-domain mutant derived from protein A used in the present invention to an immunoglobulin can vary depending on temperature under conditions of pH 5 to 9 and a temperature of lower than 60° C. As a B-domain mutants of protein A, that described in Patent Literature 1 (International Publication WO02008/143199) can be used.

The description “the binding property of the B-domain mutant derived from protein A to an immunoglobulin can vary depending on temperature under conditions of pH 5 to 9 and a temperature of lower than 60° C.” is used herein to mean that the “binding force,” binding “specificity,” and the like of the B-domain mutant derived from protein A to the immunoglobulin can vary depending on temperature under conditions of pH 5 to 9 and lower than 60° C. that do not affect the three-dimensional structure of the immunoglobulin, and that the immunoglobulin can be purified utilizing this property. Specifically, it means that when the filling of a column with the polypeptide, addition of the immunoglobulin to the column, and the washing of the column are carried out in a low temperature range, the immunoglobulin can be bound to the polypeptide, and that thereafter, the structure of the polypeptide or the like is changed by converting the low temperature range to a high temperature range, so that the immunoglobulin bound in the low ‘temperature range can be removed from the polypeptide. Specifically, the aforementioned description means that there is a difference, in terms of the binding property of the polypeptide to the immunoglobulin, between a low temperature range of, for example, 0° C. to 15° C., preferably 0° C. to 8° C. and more preferably around 5° C., and a high temperature range of, for example, 25° C. to 60° C., preferably 30° C. to 45° C., more preferably 32° C. to 38° C. and particularly preferably around 35° C. Whether or not the binding property of a candidate mutant to an immunoglobulin can vary depending on temperature can be easily confirmed by actually purifying the immunoglobulin, using the candidate mutant as a ligand for column chromatography, etc.

A specific example of the B-domain mutant derived from protein A used in the present invention is a B-domain mutant derived from protein A comprising, in a single molecule thereof, at least one amino acid sequence having homology of 60% or more with the polypeptide of SEQ ID NO: 1 (with the proviso that at least Gly at position 19 and/or Gly at position 22 are substituted with Ala or Leu in the amino acid sequence shown in SEQ ID NO: 1), wherein the binding property of the amino acid sequence to an immunoglobulin can vary depending on temperature under conditions of pH 5 to 9 and a temperature of lower than 60° C. The amino acid sequence, in which at least Gly at position 19 and/or Gly at position 22 are substituted with Ala or Leu in the amino acid sequence shown in SEQ ID NO: 1, includes mutants further comprising a substitution, deletion, addition or insertion of other amino acids, in addition to the mutations in which Gly at position 19 and/or Gly at position 22 are substituted with Ala or Leu, without changing the aforementioned mutations.

Examples of the mutation other than the mutations of positions 19 and 22 include a mutation of substituting a hydrophobic amino acid in a protein with another hydrophobic amino acid and a mutation of deleting a hydrogen bond caused by a side chain. An example of such a mutation of deleting a hydrogen bond is a substitution of Gln (in particular, for example, the Gln at position 26 exposed on the surface of the protein) into Gly. Moreover, when a hydrophilic amino acid has an extremely hydrophobic portion in the side chain thereof, if a mutation of deleting such a portion were added, the stability of the three-dimensional structure of a protein could be reduced. For example, if hydrophilic Arg having electric charge in a neutral solution is substituted with an amino acid having no (or a small number of) methylene groups with high hydrophobicity, such as Gly, the natural three-dimensional structure of the polypeptide tends to become unstable. However, it is necessary for these mutants that their binding property to an immunoglobulin can vary depending on temperature change in the range of pH 5 to 9.

The amino acid sequence of the polypeptide used in the present invention has homology of 60% or more with the polypeptide of SEQ ID NO: 1. With regard to such homology, the two amino acid sequences are identical to each other at a percentage of preferably 60% or more, more preferably 70% or more, even more preferably 80% or more, further preferably 90% or more, and particularly preferably 95% or more.

With regard to amino acid substitution, substitution of amino acids that are chemically or structurally similar to each other is preferable. Examples of the group of chemically or structurally similar amino acids include the following groups.
(Glycine, proline, alanine, and valine)
(Leucine and isoleucine)
(Glutamic acid and glutamine)
(Aspartic acid and asparagine)
(Cysteine and threonine)
(Threonine, serine, and alanine)
(Lysine and arginine)

A B-domain mutant derived from protein A comprising, in a single molecule thereof, at least one of the amino acid sequence shown in SEQ ID NO: 2 is particularly preferable.

The polypeptide used in the present invention comprises, in a single molecule thereof, at least one amino acid sequence having homology of 60% or more with the above-described polypeptide of SEQ ID NO: 1. The present polypeptide may also comprise two or more of the aforementioned amino acid sequences. The upper limit of the number of amino acid sequences comprised in the present polypeptide (hereinafter referred to as “n”) is not particularly limited. When the polypeptide is used as a ligand for affinity chromatography, the number n is preferably 6 or less, more preferably 5 or less, and particularly preferably 4 or less, taking into consideration compatibility with the size, type and the like of a support, a column, etc. used for affinity chromatography.

The polypeptide used in the present invention can be synthesized according to an ordinary method using a polypeptide synthesizer or the like. It can also be produced by producing a gene corresponding to the polypeptide and then allowing the gene to express. That is to say, a host cell is transformed with an expression vector comprising DNA encoding the amino acid sequence of the polypeptide, and the obtained transformant is then cultured, so as to produce a polypeptide.

The DNA encoding the amino acid sequence of the polypeptide is preferably inserted into an expression vector. As such an expression vector, a commercially available plasmid can be used, and the type of the expression vector is not particularly limited. For example, a pET vector (manufactured by Merck, Japan) or a pRSET vector (manufactured by Invitrogen, Japan) is preferable because these vectors are able to express a large amount of polypeptide in combination with Escherichia coli as a host. It is preferable to use an expression vector in an appropriate combination with a host cells. In the case of a pET vector and a pRSET vector, for example, Escherichia coli BL21(DE3) or C41(DE3) can be used as a host cell.

Transformation of a host cell with an expression vector can be carried out by a heat shock method, an electroporation method, etc. The transformant transformed with such an expression vector can be cultured according to an ordinary method using a suitable medium. For example, when the host is Escherichia coli, a liquid medium such as an LB medium or a 2× TY medium is used, and the transformant is preferably cultured at a temperature of generally 15° C. to 40° C., and particularly 30° C. to 37° C. It is preferable that the medium be shaken or stirred, and that ventilation or the adjustment of pH be carried out, as necessary. The expression of the polypeptide can be induced by adding isopropyl-1-β-D-galactopyranoside (IPTG), etc. to the medium.

The host cells that have expressed the polypeptide are separated from the medium according to centrifugation, filter separation, etc. The host cells are suspended in a suitable buffer solution, followed by cell disintegration. After completion of the cell disintegration, the resulting solution is subjected to centrifugation, so that the polypeptide used in the present invention can be recovered in a soluble fraction. In order to purify the polypeptide from the soluble fraction, a known polypeptide purification method can be applied. For example, the polypeptide can be purified, for example, by combining salting-out with ion exchange chromatography. Alternatively, a tag peptide existing in the N-terminus of the polypeptide can be utilized to purify the polypeptide. For example, when the tag peptide is a His tag, a purification method utilizing metal chelate affinity chromatography can be applied. In the case of using a GST tag, a purification method utilizing a glutathione-bound affinity resin can be applied. In the metal chelate affinity chromatography, Ni-NTA that is nickel-charged agarose gel, etc. can be used.

The carrier used in the present invention is not particularly limited, as long as it can be used as an adsorbent for affinity chromatography. The carrier is preferably a particulate matrix for chromatography, or a membrane (more preferably, a hollow fiber membrane). When the carrier is a particulate carrier, the mean particle diameter of the carrier is preferably 20 to 200 μm.

The raw material for the carrier is not particularly limited. As a raw material for a membranous carrier, a polymeric material capable of forming a porous membrane can be used. Examples of such a raw material that can be used herein include: olefin resins such as polyethylene and polypropylene; polyester resins such as polyethylene terephthalate and polyethylene terenaphthalate; polyamide resins such as nylon 6 and nylon 66; fluorine-containing resins such as polyvinylidene fluoride and polychlorotrifluoroethylene; and non-crystalline resins such as polystyrene, polysulfone, polyether sulfone, and polycarbonate. As raw materials for particulate matrix for chromatography, glass, silica, polystyrene resin, methacrylic resin, crosslinked agarose, crosslinked dextran, crosslinked polyvinyl alcohol, crosslinked cellulose, and the like can be used. Among others, crosslinked polyvinyl alcohol and crosslinked cellulose are preferable because they have high hydrophilicity and are able to suppress adsorption of mpure components.

A coupling group can be introduced into the above-described carrier. Examples of such a coupling group include a carboxyl group activated by N-hydroxysuccinimide (NHS), a carboxyl group, a cyanogen bromide activated group, a hydroxyl group, an epoxy group, an aldehyde group, and a thiol group. A polypeptide to be immobilized on a carrier has a primary amino group. Thus, among the aforementioned coupling groups, a carboxyl group activated by NHS, a carboxyl group, a cyanogen bromide activated group, a hydroxyl group, an epoxy group, and a formyl group, which are able to bind to such a primary amino group, are preferable. In particular, a carboxyl group activated by NHS is particularly preferable because it does not require other reagents during the coupling reaction and it also promotes a quick reaction and forms a strong bond.

It is preferable to use a carrier containing a carboxyl group at a level of 400 to 600 μmol/mL resin.

The method of introducing a coupling group into a carrier is not particularly limited. In general, a spacer is introduced between a carrier and a coupling group. A coupling group can be introduced into a carrier according to an ordinary method.

A graft polymer chain having a coupling group on the terminus and/or side chain thereof may be introduced into a carrier. By introducing a graft polymer chain having a coupling group into a support, conditions can be controlled, for example, the density of coupling groups can be arbitrarily increased. A polymer chain having a coupling group may be grafted onto a carrier. Otherwise, a polymer chain having a precursor functional group that can be converted to a coupling group may be grafted onto a carrier, and thereafter, the grafted precursor functional group may be converted to a coupling group.

The method of introducing a graft polymer chain into a carrier is not particularly limited. A polymer chain may be previously prepared, and it may be then coupled to a carrier. Otherwise, by means such as a “living radical polymerization method” or a “radiation graft polymerization method,” a graft chain may be directly polymerized on a carrier. The “radiation graft polymerization method” is preferable because it does not require previous introduction of a reaction initiator into a carrier and it is applicable to various types of carriers.

As a method of immobilizing a polypeptide on a carrier, various techniques, which are well known in the present technical field and are described in publications, can be arbitrarily used. For instance, immobilization of a polypeptide on a carrier by activating a solid support by a coupling agent such as the above-described N-hydroxysuccinimide, or by a carboxyl group or a thiol group, etc. can be applied. For example, a polypeptide can be bound to a carrier via an amide bond. The binding amount of a polypeptide is not particularly limited. From the viewpoint of the binding capacity of an immunoglobulin, a polypeptide binds to a carrier at a level of preferably 20 mg/mL resin or more, and more preferably 40 mg/mL resin or more.

In the adsorbent of the present invention, the maximum binding capacity of the immunoglobulin is preferably 20 mg/mL resin or more, and more preferably 40 mg/mL resin or more.

The present invention further provides a method for purifying an immunoglobulin, which comprises allowing a sample containing the immunoglobulin to come into contact with the adsorbent of the present invention.

The immunoglobulin used as a purification target may be either an immunoglobulin derived from living bodies or cultured cells, or an immunoglobulin artificially synthesized by imitating the structure of the aforementioned immunoglobulin. It may also be either a monoclonal antibody or a polyclonal antibody. Moreover, the immunoglobulin may also be either an immunoglobulin produced by chimerization (e.g., humanization) of an immunoglobulin derived from a non-human animal, or an immunoglobulin produced by complete humanization. Furthermore, the immunoglobulin used as a purification target may also be a phage antibody consisting only of a VH chain that is a heavy chain variable region of a monoclonal antibody and a VL chain that is a light chain variable region thereof.

In the present invention, an immunoglobulin can be eluted by temperature change, using the adsorbent of the present invention under conditions of pH 5 to 9 and a temperature of lower than 60° C. In the present invention, the control of the temperature is required. An example of a method of controlling the temperature is a method comprising disposing a circulation jacket around an affinity chromatographic column, such that circulating water or the like is allowed to directly come into contact with the circumference of the affinity chromatographic column, and then controlling the temperature of the circulating water or the like, so as to control the temperature in the column.

First, the temperature of a heating medium, such as water circulating in the jacket, is controlled to a temperature of 0° C. to 15° C., preferably 0° C. to 10° C., and more preferably 5° C., so that the temperature in the column can be set at the same temperature as described above. Thereafter, a sample solution containing an immunoglobulin is injected into the column that has been equilibrated with a suitable buffer solution with neutral pH, and then, substances that do not bind to the column are completely removed from the column, using a washing buffer solution (with neutral pH). The temperature of each of the buffer solution for equilibration, the injected sample solution, and the washing buffer solution is preferably maintained to be a desired temperature.

The immunoglobulin bound to an affinity ligand can be recovered by stabilizing the temperature in the column at 30° C. to 45° C., preferably 32° C. to 38° C., and more preferably around 37° C., and then injecting a neutral buffer solution used for elution that is maintained at the same temperature to the column in the same manner as described above.

The present invention will be described more in detail in the following examples. However, these examples are not intended to limit the scope of the present invention.

### EXAMPLES

### Example 1

(Preparation of Template Plasmid used for Site-Directed Mutagenesis)

A dsDNA was chemically synthesize wherein an NcoI recognition sequence (CCATGG) was added to the 5’-terminal side of an insertion gene (SEQ ID NO: 3) encoding a polypeptide consisting of a histidine tag sequence, a linker sequence (SEQ ID NO: 5) and repeat sequences of a temperature-responsive protein A, and a BamHI recognition sequence (GGATCC) was added to the 3′-terminal side thereof. Both ends of the synthesized DNA were cleaved with the restriction enzymes NcoI and BamHI, and the cleavage was then subjected to agarose gel electrophoresis. The reaction product was purified using QIAquick Gel Extraction Kit (manufactured by Qiagene, Japan), and the resultant was used as an insertion gene. An expression vector was prepared by cleaving the cloning site of the plasmid pET28b(+) (manufactured by Merck, Japan) with the restriction enzymes NcoI and BamHI and ligating the aforementioned insertion gene by T4 DNA ligase.

### (Transformation and Amplification of Template Plasmid)

Using the aforementioned expression vector, XL1-blue competent cells (manufactured by NIPPON GENE, CO., LTD., Japan) were transformed by a heat shock method. The reaction product was allowed to grow on an LB medium plate containing 50 μg/mL kanamycin for 18 hours. Colonies appearing on the plate were seeded in an LB liquid medium containing 50 μg/mL kanamycin, and they were then allowed to grow for 18 hours, thereby obtaining an Escherichia coli clone transformed by the aforementioned expression vector.

### (Purification of Template Plasmid)

Using QIAprep Spin miniprep kit (manufactured by Qiagene, Japan), a template plasmid used for site-directed mutagenesis was purified from the aforementioned Escherichia coli strain.

(Preparation of Expression Vector containing Mutant Polypeptide having Different Linker Sequence, and Expression)

A mutant polypeptide having a different linker sequence was produced by performing site-directed mutagenesis on the aforementioned template plasmid according to an Inverse PCR method using KOD plus Mutagenesis Kit (Toyobo Co., Ltd., Japan). After completion of the Inverse PCR, the methylated template plasmid was digested by DpnI. Thereafter, the DNA fragment that had been self-ligated using T4 DNA ligase was used as an expression vector for a mutant polypeptide having a different linker sequence. The amino acid sequence of a linker sequence portion of the produced mutant polypeptide is shown in SEQ ID NO: 5. Using an expression vector of the obtained mutant polypeptide, the E. coli BL21(DE3) strain was transformed, so as to obtain Transformant 1 expressing a mutant polypeptide.

TABLE 1
SEQ ID NO: 3

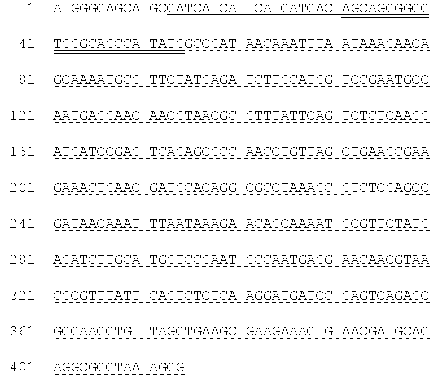

The single underlined portion indicates a His tag sequence, the double underlined portion indicates a linker sequence, and the dotted line portion indicates a sequence encoding temperature-responsive protein A.

TABLE 2

| SEQ | | |
|---|---|---|
| ID | Amino acid sequence | |
| NO: | of linker sequence | Remarks |
| 4 | S S G L V P R G S H | Comparative example |
| | M | |
| 5 | S S G L G S H M | The present invention |
| 6 | S S G L S H M | The present invention |
| 7 | S S G L S S H M | The present invention |
| 8 | S S G L S S R H M | The present invention |
| 9 | S S G L S S R G H M | The present invention |
| 10 | S S G L S S R G S H | The present invention |
| | M | |
| 11 | S S G L S S R G S S | The present invention |
| | H M | |
| 12 | S S G L H M | Comparative example |
| 13 | S S G L S S R G S S | Comparative example |
| | G H M | |
| 14 | S S G L S S R G S S | Comparative example |
| | G S H M | |
| 15 | S S G S S G S G S H | The present invention |
| | M | |
| 16 | S S G S S G S G S S | The present invention |
| | M | |

### (Confirmation of Expression Level of Mutant)

Transformant 1 expressing a mutant polypeptide was allowed to grown on an LB medium plate containing 50 μg/mL kanamycin at 37° C. for 16 hours. Thereafter, one colony was selected from the appearing colonies, and it was then seeded in an LB liquid medium containing 50 μg/mL kanamycin, followed by performing a shaking culture at 37° C. Five hours after initiation of the culture, IPTG was added to the culture to a final concentration of 1 mM, and the shaking culture was then continued for further 3 hours. The cell amount of the Transformant 1 was measured using a spectrophotometer with turbidity at a wavelength of 600 nm. The obtained value was found to be 14.8.

The cell mass was recovered from the obtained culture solution of Transformant 1 by centrifugation, and it was then suspended in 10 mM Tris-HCl (pH 8.0). To this suspension, lysozyme was added, and the obtained mixture was then treated at 15° C. for 30 minutes. Thereafter, the cell mass was disintegrated by a freezing-thawing method, and a mutant polypeptide was then recovered in a supernatant by centrifugation.

The expression level of each mutant polypeptide contained in the obtained supernatant was measured by HPLC. The expression level was found to be 1.13 mg/mL.

### Examples 2 to 9

Site-directed mutagenesis, preparation of transformants, and confirmation of expression levels were carried out in the same manner as that of Example 1, with the exception that the linker sequences of the mutant polypeptides were changed to those shown in SEQ ID NOS: 6 to 11, 15 and 16, so as to obtain the corresponding Transformants 2 to 7, 12, and 13. The results are shown in Table 3.

### Comparative Examples 1 to 4

Site-directed mutagenesis, preparation of transformants, and confirmation of expression levels were carried out in the same manner as that of Example 1, with the exception that the linker sequences of the mutant polypeptides were changed to those shown in SEQ ID NO: 4 and SEQ ID NOS: 12 to 14, so as to obtain the corresponding Transformants 10 to 13. The results are shown in Table 3.

TABLE 3

| | | Sequence | Cell amount | Expression |
|---|---|---|---|---|
| | | number of | (turbidity at | level |
| | Transformant | linker sequence | 600 nm) | (mg/ml) |
| Example 1 | 1 | 5 | 14.8 | 1.13 |
| Example 2 | 2 | 6 | 13.9 | 0.70 |
| Example 3 | 3 | 7 | 14.3 | 0.72 |
| Example 4 | 4 | 8 | 15.2 | 0.80 |
| Example 5 | 5 | 9 | 15.1 | 0.91 |
| Example 6 | 6 | 10 | 14.8 | 0.93 |
| Example 7 | 7 | 11 | 14.4 | 0.86 |
| Example 8 | 8 | 15 | 14.6 | 0.69 |
| Example 9 | 9 | 16 | 14.3 | 0.61 |
| Comp. Ex. 1 | 10 | 4 | 14.2 | 0.71 |
| Comp. Ex. 2 | 11 | 12 | 13.2 | 0.51 |
| Comp. Ex. 3 | 12 | 13 | 13.8 | 0.59 |
| Comp. Ex. 4 | 13 | 14 | 12.1 | 0.48 |

### Example 10

### (Large Scale Culture of Transformant 1 and Confirmation of Stability)

Transformant 1 of Example 1 was allowed to grow on an LB medium plate containing 50 μg/mL kanamycin at 37° C. for 16 hours. Thereafter, one colony was selected from the appearing colonies, and it was then seeded in an LB liquid medium containing 50 μg/mL kanamycin, followed by performing a shaking culture at 37° C. for 7 hours. Thereafter, 0.5 mL of the obtained culture solution was seeded in a 5-L pressurized aeration-agitation culture tank (the amount of the culture medium: 3 L; the composition of the medium: 2% glucose, 0.1% lactose monohydrate, 0.5% yeast extract, 1.0% peptone, and 0.5% NaCl), and an aeration agitation culture was then carried out at 37° C. for 16 hours. The cell amount was measured, the cell mass was then disintegrated, and the expression level of a mutant polypeptide was then measured in the same manner as that of Example 1. The cell amount was found to be 35 with turbidity at a wavelength of 600 nm, and the expression level of the mutant polypeptide was found to be 2.3 g/L per culture solution (Table 4). The obtained cell disruption solution was left at a temperature of 10° C. for 24 hours, and the concentration of the mutant polypeptide was then measured again. As a result, it was found to be 2.3 g/L (Table 4).

### Examples 11 to 18

The large scale culture of Transformants 2 to 9 and confirmation of their stability were carried out in the same manner as that of Example 10, with the exception that Transformants 2 to 9 were each used instead of Transformant 1. The results are shown in Table 4.

### Comparative Example 5

### (Large Scale Culture of Transformant 10 and Confirmation of Stability)

The culture was carried out in the same manner as that of Example 10 with the exception that Transformant 10 was used. The cell amount was found to be 32 with turbidity at a wavelength of 600 nm, and the expression level of a mutant polypeptide was found to be 1.2 g/L per culture solution (Table 4). The obtained cell disruption solution was left at a temperature of 10° C. for 24 hours, and the concentration of the mutant polypeptide was then measured again in the same manner as that of Example 10. As a result, the concentration of the mutant polypeptide was found to be 0.9 g/L (Table 4). As a result of confirmation by SDS-PAGE, a band with a lower molecular weight than the band of the mutant polypeptide appeared.

### Comparative Examples 6 to 8

The large scale culture of Transformants 11 to 13 and confirmation of their stability were carried out in the same manner as that of Comparative Example 5, with the exception that Transformants 11 to 13 were each used instead of Transformant 10. The results are shown in Table 4.

TABLE 4

| | | Sequence | | Concentration | |
|---|---|---|---|---|---|
| | | number | | of mutant | |
| | | of | | polypeptide | |
| | Trans- | linker | Turbidity | Expression | After leaving |
| | formant | sequence | at 600 nm | level | for 24 hours |
| Example 10 | 1 | 5 | 35 | 2.3 | 2.3 |
| Example 11 | 2 | 6 | 33 | 2.0 | 2.0 |
| Example 12 | 3 | 7 | 35 | 2.1 | 2.1 |
| Example 13 | 4 | 8 | 37 | 1.9 | 1.9 |
| Example 14 | 5 | 9 | 33 | 2.0 | 2.0 |
| Example 15 | 6 | 10 | 36 | 2.2 | 2.2 |
| Example 16 | 7 | 11 | 34 | 2.1 | 2.1 |
| Example 17 | 8 | 15 | 34 | 1.8 | 1.8 |
| Example 18 | 9 | 16 | 35 | 1.9 | 1.9 |
| Comparative | 10 | 4 | 32 | 1.2 | 0.9 |
| Example 5 | | | | | |
| Comparative | 11 | 12 | 33 | 1.4 | 1.0 |
| Example 6 | | | | | |
| Comparative | 12 | 13 | 29 | 0.9 | 0.7 |
| Example 7 | | | | | |
| Comparative | 13 | 14 | 30 | 0.9 | 0.6 |
| Example 8 | | | | | |

### Example 19

(Purification of Mutant Polypeptide from Culture Solution of Transformant 1)

The cell disruption solution containing a mutant polypeptide, which was obtained in Example 10, was subjected to centrifugation to obtain a supernatant containing the mutant polypeptide. The obtained supernatant was adsorbed on a Ni-Sepharose CL-6B (manufactured by GE Healthcare) column, and it was then eluted with a 10 mM Tris-HCl buffer solution (pH 8.0) containing 250 mM imidazole. The eluant was further adsorbed on an anion exchange column, and was then eluted with NaCl concentration gradient, so that it could be purified. The eluted fraction from the anion exchange column was subjected to concentration and desalination using an ultrafilter membrane (fractionated molecular weight: 3000 kDa), thereby obtaining 20 mL of a concentrate of the mutant polypeptide. The amount of the mutant polypeptide contained in the concentrate was found to be 1.0 g.

### (Immobilization of Mutant Polypeptide on Crosslinked Polyvinyl Alcohol Beads)

The obtained mutant polypeptide was immobilized on crosslinked polyvinyl alcohol beads according to the following method.

### 1) Introduction of Carboxyl Group

3.0 g of succinic anhydride and 3.6 g of 4-dimethylaminopyridine were dissolved in 450 mL of toluene, and the obtained solution was used as a reaction solution. 1 g of crosslinked polyvinyl alcohol beads (mean particle diameter: 100 μm) was allowed to come into contact with the aforementioned reaction solution at 50° C., and the reaction solution was then stirred for 2 hours. Thereafter, the crosslinked polyvinyl alcohol beads were washed with dehydrated isopropyl alcohol. The amount of carboxyl group introduced was measured. As a result, it was found to be 443 μmol/mL per volume of beads.

### 2) Column Packing

An empty column (Tricorn 5/20, manufactured by GE Healthcare) was filled with the aforementioned crosslinked polyvinyl alcohol beads.

### 3) NHS Activation

An NHS activation reaction solution (0.07 g of NHS, 45 mL of dehydrated isopropyl alcohol, and 0.09 mL of diisopropylcarbodiimide) was supplied to the aforementioned column at a flow rate of 0.4 mL/min for 30 minutes, while heating the column to 40° C., so that carboxyl groups were activated by NHS. After completion of the reaction, the column was washed by allowing dehydrated isopropyl alcohol to pass through the column.

4) Coupling with Mutant Polypeptide

To the aforementioned NHS activated column, 2 mL of 1 mM hydrochloric acid that had been cooled on ice was supplied, so that it was substituted for dehydrated isopropyl alcohol. Subsequently, 30 mg of mutant polypeptide was dissolved in 1 mL of a coupling buffer solution (0.2 M phosphate buffer solution, 0.5 M NaCl, pH 8.3), and the obtained solution was then cooled to 2° C. The cooled solution was supplied to the column at a flow rate of 0.4 mL/min, and it was then retained for 16 hours. After a predetermined period of time had passed, a coupling buffer solution was supplied to the column, so as to wash and/or recover the mutant polypeptide that was not coupled to the NHS active group.

### 5) Blocking

10 mL of a blocking reaction solution (0.5 M ethanolamine, 0.5 M NaCl, pH 8.0) was supplied to the mutant polypeptide-coupled column, so that the residual NHS was blocked by ethanolamine. After completion of the blocking reaction, the column was washed with pure water, and it was then preserved at 4° C. in a state in which it was enclosed with 20% ethanol.

### 6) Measurement of the Maximum Binding Capacity and Dynamic Adsorption Capacity of Immunoglobulin

Using Chromatography System (AKTA FPLC, manufactured by GE Healthcare), examinations were carried out regarding adsorption and/or elution of immunoglobulin (Venoglobulin-IH blood donation, manufactured by Benesis Corporation) by temperature change. The operation to change the temperature of the column was carried out by once stopping a pump of the Chromatography System, immersing the column in a constant temperature water tank with a predetermined temperature, leaving it for 10 or more minutes, and then starting the pump of the Chromatography System again. The adsorption temperature was set at 2° C., and the elution temperature was set at 40° C. After completion of the elution by temperature change, an antibody that had not been eluted was eluted with an elution buffer solution with low pH (0.1 M citrate buffer solution, pH 3.0). The UV absorption (280 nm) of each elution fraction was measured, and the concentration of immunoglobulin was then calculated according to the formula as shown below, so that the maximum binding capacity of the immunoglobulin was calculated.

Immunoglobulin concentration (mg/mL)=absorbance at 280 nm/14×10

Maximum binding capacity (mg/mL)=Immunoglobulin concentration of temperature elution fraction×liquid amount of temperature elution fraction/volume of beads

The dynamic adsorption capacity of the immunoglobulin was calculated based on the elution volume at a 10% breakthrough point of the obtained breakthrough curve.

### (Results)

The maximum binding capacity of the immunoglobulin was found to be 34.0 mg/mL per volume of beads, and the dynamic adsorption capacity of the immunoglobulin was found to be 19.9 mg/mL per volume of beads (Table 5).

### Example 20

The measurement was carried out under the same conditions as those of Example 19, with the exception that the mean particle diameter of crosslinked polyvinyl alcohol beads was set at 60 μm. The maximum binding capacity of the immunoglobulin was found to be 47.0 mg/mL per volume of beads, and the dynamic adsorption capacity of the immunoglobulin was found to be 26.0 mg/mL per volume of beads (Table 5).

### Example 21

The measurement was carried out under the same conditions as those of Example 19, with the exception that crosslinked cellulose beads were used instead of crosslinked polyvinyl alcohol beads. The maximum binding capacity of the immunoglobulin was found to be 18.9 mg/mL per volume of beads, and the dynamic adsorption capacity of the immunoglobulin was found to be 2.9 mg/mL per volume of beads (Table 5).

### Example 22

The measurement was carried out under the same conditions as those of Example 19, with the exception that crosslinked agarose beads were used instead of crosslinked polyvinyl alcohol beads. The maximum binding capacity of the immunoglobulin was found to be 18.0 mg/mL per volume of beads, and the dynamic adsorption capacity of the immunoglobulin was found to be 6.1 mg/mL per volume of beads (Table 5).

### Example 23

Using the concentrate of the mutant polypeptide obtained in Example 19, the mutant polypeptide was immobilized on a hollow fiber.

### 1) Surface Graft Polymerization

20 g of GMA was dissolved in 180 mL of methanol, and it was then bubbled with nitrogen for 30 minutes. The obtained solution was used as a reaction solution. 2 g of hollow fiber made of polyethylene (inner diameter: 2.0 mm; outer diameter: 3.0 mm; mean pore diameter: 0.25 μm) was irradiated with γ-ray (200 kGy) using cobalt 60 as a radiation source in a nitrogen atmosphere, while it was cooled with dry ice to −60° C. After completion of the irradiation, the hollow fiber was left at rest under a reduced pressure of 13.4 pa or less for 5 minutes. Thereafter, the resulting hollow fiber was allowed to come into contact with 20 mL of the aforementioned reaction solution at 40° C., and it was then left at rest for 16 hours. Thereafter, the hollow fiber was washed with ethanol, and was then vacuum-dried in a vacuum dryer.

### 2) Conversion of Epoxy Group to Diol Group

The surface graft-polymerized hollow fiber was added into 0.5 mol/L sulfuric acid, and a reaction was then carried out at 80° C. for 2 hours, so that epoxy groups remaining in the graft chain were conserved to diol groups. After completion of this reaction, the hollow fiber was washed with pure water. Thereafter, the membrane was washed with ethanol, and was then vacuum-dried in a vacuum dryer.

### 3) Introduction of Carboxyl Group

The hollow fiber, in which the epoxy groups had been converted to diol groups, was immersed in a reaction solution prepared by dissolving 3.0 g of succinic anhydride and 3.6 g of 4-dimethylaminopyridine in 900 mL of toluene, and a reaction was then carried out at 40° C. for 60 minutes, so that a carboxyl group was introduced into the graft chain. After completion of this reaction, the hollow fiber was washed with ethanol, and was then vacuum-dried in a vacuum dryer.

### 4) NHS Activation

While a modularized hollow fiber (a single module of hollow fiber; effective fiber length: 4 cm) was heated to 40° C., an NHS activation reaction solution (0.07 g of NHS, 45 mL of dehydrated isopropyl alcohol, and 0.09 mL of diisopropylcarbodiimide) was supplied to the fiber at a flow rate of 0.4 mL/min for 60 minutes, so that the carboxyl group was activated by NHS. After completion of the reaction, while cooling the hollow fiber module on ice, dehydrated isopropyl alcohol was supplied to the hollow fiber module at a flow rate of 0.4 mL/min for 60 minutes, so as to wash it. The washed hollow fiber module was preserved at 4° C. in a state in which it was enclosed with dehydrated isopropyl alcohol.

### 5) Coupling of Mutant Polypeptide

To the hollow fiber module, in which the carboxyl group had been activated by NHS, 10 mL of 1 mmol/L hydrochloric acid that had been cooled on ice was supplied, so that it was substituted for the dehydrated isopropyl alcohol serving as a preservative solution. Subsequently, 20 mg of the mutant polypeptide obtained in Example 19 was dissolved in 7 mL of a coupling buffer solution (0.2 mol/L phosphate buffer solution, 0.5 mol/L NaCl, pH 8.3), and the obtained solution was then cooled to 2° C. The resulting solution was supplied to the hollow fiber at a flow rate of 0.4 mL/min. The permeated solution was continuously added to a solution to be supplied, so that the solution was circulated for 16 hours. The temperature during the coupling reaction was kept at 2° C. by keeping the module at 2° C. even during the circulation. After a predetermined period of time had passed, the coupling buffer solution was supplied to the hollow fiber module, so that the mutant peptide that had not been coupled to the NHS active group could be washed and recovered.

### 6) Blocking

10 mL of a blocking reaction solution (0.5 M ethanolamine, 0.5 M NaCl, pH 8.0) was supplied to the mutant polypeptide-coupled hollow fiber module, and it was then left at room temperature for 30 minutes, so that the residual NHS was blocked by ethanolamine After completion of the blocking reaction, the hollow fiber module was washed with pure water, and it was then preserved at 4° C. in a state in which it was enclosed with 20% ethanol.

### 6) Measurement of the Maximum Binding Capacity and Dynamic Adsorption Capacity of Immunoglobulin

Using Chromatography System (AKTA FPLC, manufactured by GE Healthcare), examinations were carried out regarding adsorption and/or elution of immunoglobulin (Venoglobulin-IH blood donation, manufactured by Benesis Corporation) by temperature change in the same manner as that of Example 19. The concentration of immunoglobulin was calculated according to the formula as shown below, so that the maximum binding capacity of the immunoglobulin was calculated.

Immunoglobulin concentration (mg/mL)=absorbance at 280 nm/14×10

Maximum binding capacity (mg/mL)=Immunoglobulin concentration of temperature elution fraction×liquid amount of temperature elution fraction/volume of membrane

### (Results)

The maximum binding capacity of the immunoglobulin was found to be 15.3 mg/mL per volume of membrane (Table 5).

### Example 24 to 31

A mutant polypeptide was purified from the culture solution, it was then immobilized on crosslinked polyvinyl alcohol beads, and the maximum binding capacity and dynamic adsorption capacity of the immunoglobulin were then measured under the same conditions as those of Example 19, with the exceptions that Transformants 2 to 8 were each used, and that the mean particle diameter of the crosslinked polyvinyl alcohol beads was set at 60 μm. The results are shown in Table 5.

### Comparative Examples 9 to 12

A mutant polypeptide was purified from the culture solution, it was then immobilized on crosslinked polyvinyl alcohol beads, and the maximum binding capacity and dynamic adsorption capacity of the immunoglobulin were then measured under the same conditions as those of Example 19, with the exceptions that Transformants 10 to 13 were each used, and that the mean particle diameter of the crosslinked polyvinyl alcohol beads was set at 60 μm. The results are shown in Table 5.

TABLE 5

| | | Sequence | | | |
|---|---|---|---|---|---|
| | | number | | Maxi- | |
| | | of | | mum | Dynamic |
| | Trans- | linker | | binding | binding |
| | formant | sequence | Carrier | capacity | capacity |
| Example 19 | 1 | 5 | Crosslinked PVA | 34 | 19.9 |
| | | | (100 um) | | |
| Example 20 | 1 | 5 | Crosslinked PVA | 47 | 26 |
| | | | (60 um) | | |
| Example 21 | 1 | 5 | Crosslinked | 18.9 | 2.9 |
| | | | cellulose | | |
| Example 22 | 1 | 5 | Crosslinked | 18 | 6.1 |
| | | | agarose | | |
| Example 23 | 1 | 5 | Hollow fiber | 15.3 | — |
| | | | membrane | | |
| Example 24 | 2 | 6 | Crosslinked PVA | 43 | 22 |
| | | | (60 um) | | |
| Example 25 | 3 | 7 | Crosslinked PVA | 46 | 24 |
| | | | (60 um) | | |
| Example 26 | 4 | 8 | Crosslinked PVA | 45 | 24 |
| | | | (60 um) | | |
| Example 27 | 5 | 9 | Crosslinked PVA | 40 | 22 |
| | | | (60 um) | | |
| Example 28 | 6 | 10 | Crosslinked PVA | 41 | 24 |
| | | | (60 um) | | |
| Example 29 | 7 | 11 | Crosslinked PVA | 44 | 25 |
| | | | (60 um) | | |
| Example 30 | 8 | 15 | Crosslinked PVA | 42 | 20 |
| | | | (60 um) | | |
| Example 31 | 9 | 16 | Crosslinked PVA | 41 | 21 |
| | | | (60 um) | | |
| Comparative | 10 | 4 | Crosslinked PVA | 33 | 12.5 |
| Example 9 | | | (60 um) | | |
| Comparative | 11 | 12 | Crosslinked PVA | 35 | 13.7 |
| Example 10 | | | (60 um) | | |
| Comparative | 12 | 13 | Crosslinked PVA | 33 | 13.6 |
| Example 11 | | | (60 um) | | |
| Comparative | 13 | 14 | Crosslinked PVA | 34 | 12.7 |
| Example 12 | | | (60 um) | | |

1. An adsorbent consisting of a carrier, to which a polypeptide comprising a tag peptide, a linker sequence, and a B-domain mutant derived from protein A from the N-terminal side thereof binds, wherein
the linker sequence is an amino acid sequence that does not comprise a Val-Pro-Arg sequence and is composed of 7 to 12 amino acid residues; and
the binding property of the B-domain mutant derived from protein A to an immunoglobulin can vary depending on temperature under conditions of pH 5 to 9 and a temperature of lower than 60° C.

2. The adsorbent according to claim 1, wherein the linker sequence comprises 1 to 4 glycine residues and 3 to 7 serine residues.

3. The adsorbent according to claim 1, wherein the linker sequence comprises a methionine residue.

4. The adsorbent according to claim 1, wherein the linker sequence comprises a leucine residue.

5. The adsorbent according to claim 1, wherein the linker sequence comprises a histidine residue.

6. The adsorbent according to claim 1, wherein the linker sequence is any one of:
an amino acid sequence composed of a glycine residue, a serine residue and a methionine residue;
an amino acid sequence composed of a glycine residue, a serine residue, a methionine residue and a histidine residue;
an amino acid sequence composed of a glycine residue, a serine residue, a methionine residue, a histidine residue and a leucine residue; and
an amino acid sequence composed of a glycine residue, a serine residue, a methionine residue, a histidine residue, a leucine residue and an arginine residue.

7. The adsorbent according to claim 1, wherein the linker sequence is an amino acid sequence shown by Ser-Ser-Gly-(Xaa)n-Met (wherein n represents an integer of 3 to 8, and an n number of Xaa each independently represents a glycine residue, a serine residue, a histidine residue, a leucine residue or an arginine residue).

8. The adsorbent according to claim 1, wherein the linker sequence is an amino acid sequence shown by Ser-Ser-Gly-Leu-(Xbb)m-His-Met (wherein m represents an integer of 1 to 6, and an m number of Xbb each independently represents a glycine residue, a serine residue or an arginine residue).

9. The adsorbent according to claim 1, wherein the tag peptide is a 6× histidine tag.

10. The adsorbent according to claim 1, wherein the B-domain mutant derived from protein A comprises, in a single molecule thereof, at least one amino acid sequence having homology of 60% or more with the polypeptide of SEQ ID NO: 1 (with the proviso that at least Gly at position 19 and/or Gly at position 22 are substituted with Ala or Leu in the amino acid sequence shown in SEQ ID NO: 1), wherein the binding property of the amino acid sequence to an immunoglobulin can vary depending on temperature under conditions of pH 5 to 9 and a temperature of lower than 60° C.

11. The adsorbent according to claim 1, wherein the B-domain mutant derived from protein A comprises, in a single molecule thereof, at least one of the amino acid sequence shown in SEQ ID NO: 2.

12. The adsorbent according to claim 1, wherein the carrier is a particulate matrix for chromatography.

13. The adsorbent according to claim 1, wherein the mean particle diameter of the carrier is 20 to 200 μm.

14. The adsorbent according to claim 1, wherein the carrier is composed of a crosslinked polymer of polyvinyl alcohol.

15. The adsorbent according to claim 1, wherein the polypeptide is bound to the carrier via an amide bond.

16. The adsorbent according to claim 1, wherein the polypeptide binds to the carrier at a level of 20 mg/mL resin or more.

17. The adsorbent according to claim 1, wherein the maximum binding capacity of the immunoglobulin is 20 mg/mL resin or more.

18. The adsorbent according to claim 1, wherein the carrier comprises a carboxyl group at a level of 400 to 600 μmol/mL resin.

19. The adsorbent according to claim 1, wherein the carrier is a membrane.

20. The adsorbent according to claim 19, wherein the membrane is a hollow fiber membrane.

21. The adsorbent according to claim 19, wherein the membrane is produced from a base membrane into which a graft polymer chain is introduced.

22. A method for purifying an immunoglobulin, which comprises allowing a sample containing the immunoglobulin to come into contact with the adsorbent according to claim 1.


SEQUENCE LISTING (16 sequences)

SEQ ID NO: 1 - protein, 59 aa, Artificial
  B domain mutant (BGG)
ADNKFNKEQQNAFYEILHGPNGNEEQRNAFIQSLKDDPSQSANLLAEAKKLNDAQAPKA

SEQ ID NO: 2 - protein, 59 aa, Artificial
  B domain mutant (BGG)
ADNKFNKEQQNAFYEILHGPNANEEQRNAFIQSLKDDPSQSANLLAEAKKLNDAQAPKA

SEQ ID NO: 3 - DNA, 414 bp, Artificial
  Chemically synthesized DNA
atgggcagcagccatcatcatcatcatcacagcagcggcctgggcagccatatggccgataacaaatttaataaagaacagcaaaatgcgttctatgagatcttgcatggtccgaatgccaatgaggaacaacgtaacgcgtttattcagtctctcaaggatgatccgagtcagagcgccaacctgttagctgaagcgaagaaactgaacgatgcacaggcgcctaaagcgtctcgagccgataacaaatttaataaagaacagcaaaatgcgttctatgagatcttgcatggtccgaatgccaatgaggaacaacgtaacgcgtttattcagtctctcaaggatgatccgagtcagagcgccaacctgttagctgaagcgaagaaactgaacgatgcacaggcgcctaaagcg

SEQ ID NO: 4 - protein, 11 aa, Artificial
  Linker sequence
SSGLVPRGSHM

SEQ ID NO: 5 - protein, 8 aa, Artificial
  Linker sequence
SSGLGSHM

SEQ ID NO: 6 - protein, 7 aa, Artificial
  Linker sequence
SSGLSHM

SEQ ID NO: 7 - protein, 8 aa, Artificial
  Linker sequence
SSGLSSHM

SEQ ID NO: 8 - protein, 9 aa, Artificial
  Linker sequence
SSGLSSRHM

SEQ ID NO: 9 - protein, 10 aa, Artificial
  Linker sequence
SSGLSSRGHM

SEQ ID NO: 10 - protein, 11 aa, Artificial
  Linker sequence
SSGLSSRGSHM

SEQ ID NO: 11 - protein, 12 aa, Artificial
  Linker sequence
SSGLSSRGSSHM

SEQ ID NO: 12 - protein, 6 aa, Artificial
  Linker sequence
SSGLHM

SEQ ID NO: 13 - protein, 13 aa, Artificial
  Linker sequence
SSGLSSRGSSGHM

SEQ ID NO: 14 - protein, 14 aa, Artificial
  Linker sequence
SSGLSSRGSSGSHM

SEQ ID NO: 15 - protein, 11 aa, Artificial
  Linker sequence
SSGSSGSGSHM

SEQ ID NO: 16 - protein, 11 aa, Artificial
  Linker sequence
SSGSSGSGSSM